SEQUENCE LISTING (16 sequences)

SEQ ID NO: 1 - DNA, 20 bp, Artificial sequence
  Synthetic primer
acccagaagactgtggatgg

SEQ ID NO: 2 - DNA, 20 bp, Artificial sequence
  Synthetic primer
tctagacggcaggtcaggtc

SEQ ID NO: 3 - DNA, 20 bp, Artificial sequence
  Synthetic primer
gcctaaggacctggtgaagc

SEQ ID NO: 4 - DNA, 20 bp, Artificial sequence
  Synthetic primer
ttgacgaagcactcgttgag

SEQ ID NO: 5 - DNA, 20 bp, Artificial sequence
  Synthetic primer
tgcccagaaaatgaaaaagg

SEQ ID NO: 6 - DNA, 20 bp, Artificial sequence
  Synthetic primer
gtgtatgtggcaatgcgttc

SEQ ID NO: 7 - DNA, 20 bp, Artificial sequence
  Synthetic primer
acagtggccacctacaaagg

SEQ ID NO: 8 - DNA, 20 bp, Artificial sequence
  Synthetic primer
ccgagatggggttgataatg

SEQ ID NO: 9 - DNA, 20 bp, Artificial sequence
  Synthetic primer
cagtgggagacctcgagaag

SEQ ID NO: 10 - DNA, 20 bp, Artificial sequence
  Synthetic primer
tccctcggaacatcagaaac

SEQ ID NO: 11 - DNA, 20 bp, Artificial sequence
  Synthetic primer
gagaactttgccgttgaagc

SEQ ID NO: 12 - DNA, 20 bp, Artificial sequence
  Synthetic primer
gcttcctgtaggtggcaatc

SEQ ID NO: 13 - DNA, 20 bp, Artificial sequence
  Synthetic primer
cctccctgtcagatgaggac

SEQ ID NO: 14 - DNA, 20 bp, Artificial sequence
  Synthetic primer
ccaggctgaggtattccttg

SEQ ID NO: 15 - DNA, 21 bp, Artificial sequence
  Synthetic oligo
cctgagcgagcagaattacta

SEQ ID NO: 16 - DNA, 21 bp, Artificial sequence
  Synthetic oligo
gcgggagatgttcaactccca

(19) United States
(12) Patent Application Publication
(10) Pub. No.: US 2014/0275201 A1
(43) Pub. Date: Sep. 18, 2014

(54) IDENTIFICATION OF CANCER STEM CELL MARKERS AND USE OF INHIBITORS THEREOF TO TREAT CANCER

(71) Applicant: BOARD OF REGENTS, THE UNIVERSITY OF TEXAS SYSTEM, Austin, TX (US)

(72) Inventors: Sendurai A. MANI, Houston, TX (US); Brett G. HOLLIER, Brisbane (AU); Kurt W. EVANS, Houston, TX (US); Steven J. WERDEN, Pearland, TX (US); Tapasree Roy SARKAR, Houston, TX (US); Agata TINNIRELLO, Houston, TX (US)

(73) Assignee: BOARD OF REGENTS, THE UNIVERSITY OF TEXAS SYSTEM, Austin, TX (US)

(21) Appl. No.: 14/205,770

(22) Filed: Mar. 12, 2014

Related U.S. Application Data
(60) Provisional application No. 61/777,528, filed on Mar. 12, 2013.

(51) Int. Cl. G01N 33/574; A61K 45/06; C12Q 1/68; A61K 31/4045

(52) U.S. Cl. CPC G01N 33/5748; A61K 31/4045; A61K 45/06; C12Q 1/6886; G01N 33/57415
USPC 514/414; 435/6.11; 506/9; 435/7.23; 435/7.1; 435/7.92

(57) ABSTRACT

The present invention provides biomarkers of cancer stem cells, as well as cells that have undergone EMT. In addition, the present invention provides methods of treating patients determined to comprise cancer stem cells with PDGFR-β inhibitors to eliminate the cancer stem cells. Methods of predicting sensitivity to PDGFR-β inhibitors, monitoring efficacy of treatment, and determining a prognosis are also provided.

This application claims the benefit of U.S. Provisional Patent Application No. 61/777,528, filed Mar. 12, 2013, the entirety of which is incorporated herein by reference.

The invention was made with government support under Grant No. R01 CA155243-01 awarded by the National Institutes of Health. The government has certain rights in the invention.

### INCORPORATION OF SEQUENCE LISTING

The sequence listing that is contained in the file named “UTSCP1209US_ST25.txt”, which is 4 KB (as measured in Microsoft Windows®) and was created on Mar. 12, 2014, is filed herewith by electronic submission and is incorporated by reference herein.

### BACKGROUND OF THE INVENTION

1. Field of the Invention

The present invention relates generally to the field of cancer biology. More particularly, it concerns the detection and inhibition of cancer stem cells.

2. Description of Related Art

Despite the initial effectiveness of conventional therapies, recurrence and the emergence of metastases are major causes of therapeutic failure in cancer patients. These therapies are believed to target the differentiated and proliferative cells that comprise the bulk of the tumor. The relatively high relapse rate of patients with aggressive forms of breast cancer, including the recently identified triple-negative claudin-low/basal B subtype (for brevity referred to as claudin-low throughout), is attributed to a small population of cancer stem cells (CSCs) residing within the tumor. In addition to resistance to standard therapies, CSCs are reported to have inherently greater tumor-initiating potential, which is implicated in tumor relapse (Creighton et al., 2009), driving primary tumor growth as well as the seeding and establishment of metastases (Abraham et al., 2005; Al-Hajj et al., 2003; Ginestier et al., 2007; Liu et al., 2007; Sheridan et al., 2006; Shackleton et al., 2009).

The epithelial-to-mesenchymal transition (EMT) program has recently been linked to the generation of breast CSC-like cells (Mani et al., 2008) and has well documented roles in promoting an invasive and metastatic phenotype (Thiery, 2002; Yang and Weinberg, 2008). EMT was initially characterized as an important program during normal embryonic development (Hey, 1995; Thiery, 2003). However, more recent reports suggest that carcinoma cells are capable of reactivating the EMT program during tumor progression (Thiery, 2002). Similar to cells that undergo EMT during normal development, carcinoma cells that undergo EMT lose cell-cell contacts, undergo major changes in their cytoskeleton and acquire a mesenchymal-like morphology endowing them with increased invasive and migratory abilities (Hay, 1995; Thiery, 2003; Savagner et al., 1994). Several recent studies have also demonstrated that CSCs as well as cells that have undergone EMT are relatively resistant to conventional chemo- and radio-therapies (Creighton et al., 2009; Li et al., 2008; Hollier et al., 2009; Gupta et al., 2009; Fillmore and Kuperwasser, 2008; Yu et al., 2007; Woodward et al., 2007). However, it remain unclear which signaling molecules are the crucial actors in EMT, and more importantly, in CSC development. Therefore, novel targets for anti-EMT-based therapies that inhibit CSCs are greatly needed.

### SUMMARY OF THE INVENTION

Embodiments of the present invention provide therapeutic approaches that specifically target the CSC population, which can be used in combination with conventional therapies, to provide a therapeutic strategy to substantially improve cancer patient outcome. In addition, targeted therapeutics to inhibit the EMT program are contemplated to provide significant clinical benefits in treating aggressive cancers, such as aggressive breast cancers, for which current therapies are inadequate.

One embodiment of the present invention provides a method of treating a patient comprising selecting a patient determined to comprise cancer stem cells and treating the patient with an effective amount of a PDGFR-β inhibitor, thereby inhibiting the cancer stem cells. In one aspect, selecting a patient determined to comprise cancer stem cells comprises selecting a patient determined to comprise cancer cells that express an elevated level of FOXC2 or PDGFR-β relative to a reference level.

In another embodiment, the present invention provides a method of treating a patient comprising obtaining a sample of the cancer; assaying the sample for the presence of FOXC2 or PDGFR-β; identifying the patient as having a cancer that is enriched in cancer stem cells if the level of FOXC2 or PDGFR-β is elevated relative to a reference level; and treating the patient determined to have a cancer enriched in cancer stem cells with a PDGFR-β inhibitor.

In one aspect, the patient has a tumor. The patient may have a breast cancer, colon cancer, prostate cancer, or a brain tumor. The patient may have a lung cancer, head and neck cancer, esophageal cancer, tracheal cancer, liver cancer, bladder cancer, stomach cancer, pancreatic cancer, adrenal cancer, ovarian cancer, uterine cancer, cervical cancer, testicular cancer, rectal cancer, blood cancer, or skin cancer. The breast cancer may be a Claudin-low breast cancer. In another aspect, the patient may have a metastatic cancer. In another aspect, the patient may have previously undergone at least one round of anti-cancer therapy. The patient may be in remission.

In one aspect, treatment with an effective amount of a PDGFR-β inhibitor may comprising treating the patient with sunitinib, axitinib, BIBF1120, MK-2461, dovitinib, pazopanib, telatinib, CP 673451, or TSU-68. In one aspect, the PDGFR-β inhibitor is administered in conjunction with at least a second therapy, such as, for example, surgical, radiation, hormonal, cancer cell-targeted or chemotherapeutic anticancer therapy. The PDGFR-β inhibitor and the second therapy may be administered essentially simultaneously. In certain cases the PDGFR-β inhibitor may be administered before or after the second therapy.

Another embodiment of the present invention provides a method of predicting the sensitivity of a cancer in a patient to PDGFR-β inhibitors comprising obtaining a sample of the cancer and assaying to determine a level of FOXC2 or PDGFR-β expression in the sample, wherein if the level of FOXC2 or PDGFR-β is elevated relative to a reference level, then the cancer is predicted to be sensitive to a PDGFR-β inhibitor or wherein if the level of FOXC2 or PDGFR-β is not elevated relative to a reference level, then the cancer is not predicted to be sensitive to a PDGFR-β inhibitor. The method may further comprise identifying the patient as having a cancer that is sensitive to a PDGFR-β inhibitor if the level of FOXC2 or PDGFR-β is elevated relative to a reference level. Alternatively, the method may further comprise identifying the patient as having a cancer that is not sensitive to a PDGFR-β inhibitor if the level of FOXC2 or PDGFR-β is not elevated relative to a reference level.

In certain aspects, identifying a patient may further comprise reporting whether the patient has a cancer that is sensitive to a PDGFR-β inhibitor. Reporting may comprise preparing a written or oral report. Reporting may also comprise providing a report to the patient, a doctor, a hospital or an insurance provider.

Another embodiment of the present invention provides a method of treating a patient comprising selecting a patient determined to comprise a cancer that is sensitive to PDGFR-β inhibitors based on assaying for and detecting an increased expression level of FOXC2 or PDGFR-β and treating the patient with an effective amount of a PDGFR-β inhibitor, thereby inhibiting the cancer stem cells.

Another embodiment of the present invention provides a method of monitoring the efficacy of PDGFR-β treatment on a cancer comprising obtaining samples of the cancer from at least two time points during the course of treatment, assaying the FOXC2 or PDGFR-β expression level in the samples, and comparing the FOXC2 or PDGFR-β expression levels, wherein the PDGFR-β pathway inhibitor treatment is efficacious if the FOXC2 or PDGFR-β expression level decreases over the course of treatment.

Another embodiment of the present invention provides a method of identifying a cancer enriched with cancer stem cells comprising obtaining a sample of the cancer and assaying the presence of FOXC2 in the sample, wherein the cancer is enriched with cancer stem cells if FOXC2 is present. In one aspect, the sample being assayed is obtained from a metastatic site.

Another embodiment of the present invention provides a method of treating a cancer patient comprising determining if the patient has a cancer that is enriched in cancer stem cells based on assaying the presence of FOXC2 and treating the patient determined to have a cancer that is enriched in cancer stem cells with a PDGFR-β inhibitor.

Another embodiment of the present invention provides a method of selecting a drug therapy for a cancer patient comprising obtaining a sample of the cancer, assaying the presence of FOXC2 in the sample, and selecting a stem cell-specific therapy if FOXC2 if found to be present. In one aspect, the stem cell-specific therapy is a PDGFR-β inhibitor.

Another embodiment of the present invention provides a method of determining a prognosis of a cancer patient comprising obtaining a sample of the patient's cancer and assaying for the presence of FOXC2 in the sample, wherein the cancer is determined to have a poor prognosis if FOXC2 is present. In one aspect, the present embodiment further comprises treating a cancer with a poor prognosis with stem cell-specific therapy. In another aspect, the present embodiment further comprises monitoring the response to treatment by assays the FOXC2 expression level at at least two time points during treatment and comparing the expression levels, wherein the treatment is efficacious if the FOXC2 expression level decreases over time.

Another embodiment of the present invention provides a method of classifying a cancer comprising obtaining a sample of the cancer and assaying the presence of FOXC2 in the sample, wherein the cancer is determined to be metastatic of FOXC2 is present.

Another embodiment of the present invention provides a method of treating a cancer patient comprising determining the cancer patient's prognosis by assaying for the presence of FOXC2 and applying a stem cell-specific therapy if FOXC2 is present.

In some aspects of the embodiments, assaying to determine a level of FOXC2 or PDGFR-β expression may comprise determining a level of FOXC2 or PDGFR-β protein expression. The method of assaying may comprise measuring the amount of FOXC2 or PDGFR-β protein in the sample to the amount of FOXC2 or PDGFR-β protein in a control sample by contacting the samples to an antibody that binds to FOXC2 or PDGFR-β and comparing the amount of protein in the sample and the control sample. Examples of assays to measure protein expression levels include, but are not limited to, ELISA, flow cytometry, immunohistochemistry, western blot, radioimmunoassay, and immunoprecipitation.

In other aspects of the embodiments, assaying to determine a level of FOXC2 or PDGFR-β expression may comprise determining a level of FOXC2 or PDGFR-β RNA expression. The method of assaying may comprise measuring the amount of FOXC2 or PDGFR-β RNA in the sample to the amount of FOXC2 or PDGFR-β RNA in a control sample by hybridization of samples with a nucleic acid molecule that binds to FOXC2 or PDGFR-β RNA and comparing the amount of RNA in the sample and the control sample. Examples of assays to measure RNA expression levels include, but are not limited to, qRT-PCR, an array hybridization or a northern blot assay.

In some aspects of the present embodiments, determining a level of FOXC2 expression may comprise determining the expression level of one or more FOXC1-regulated genes. The expression level may be the expression level of either the mRNA or protein product of the FOXC1-regulated gene(s).

The expression of genes may be measured by a variety of techniques that are well known in the art. Quantifying the levels of the messenger RNA (mRNA) of a gene via, for example, cDNA microarray, qRT-PCR, in situ hybridization, or Northern blotting may be used to measure the expression of the biomarker. Alternatively, quantifying the levels of the protein product of a gene via, for example, ELISA, immunohistochemistry, mass spectrometry, or Western blotting may be used to measure the expression of the gene. Additional information regarding the methods discussed below may be found in Ausubel et al. (2003) or Sambrook et al. (1989). One skilled in the art will know which parameters may be manipulated to optimize detection of the mRNA or protein of interest.

As used herein, “increased expression” refers to an elevated or increased level of expression in a cancer sample relative to a suitable control (e.g., a non-cancerous tissue or cell sample, a reference standard). As used herein, “decreased expression” refers to a reduced or decreased level of expression in a cancer sample relative to a suitable control (e.g., a non-cancerous tissue or cell sample, a reference standard).

“Prognosis” refers to a prediction of how a patient will progress, and whether there is a chance of recovery. “Cancer prognosis” generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression-free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer. Prognosis also includes prediction of favorable responses to cancer treatments, such as a conventional cancer therapy.

A good or bad prognosis may, for example, be assessed in terms of patient survival, likelihood of disease recurrence or disease metastasis (patient survival, disease recurrence and metastasis may for example be assessed in relation to a defined timepoint, e.g., at a given number of years after cancer surgery (e.g., surgery to remove one or more tumors) or after initial diagnosis. In one embodiment, a good or bad prognosis may be assessed in terms of overall survival or disease free survival.

For example, “good prognosis” may refer to the likelihood that a patient afflicted with cancer will remain disease free (e.g., cancer free) or survive despite the presence of the cancer. “Poor prognosis” may be used to mean the likelihood of a relapse or recurrence of the underlying cancer or tumor, metastasis, or death. Cancer patients classified as having a “good prognosis” may remain free of the underlying cancer or tumor or survive despite the presence of cancer or tumor. For example, cancerous cells and/or tumors from a cancer may continue to exist in a patient with a good prognosis, but the patient's immune system may slow or prevent the progression or growth of the cancer, thus allowing the patient to continue to survive. In contrast, “bad prognosis” cancer patients experience disease relapse, tumor recurrence, metastasis, and death. In particular embodiments, the time frame for assessing prognosis and outcome is, for example, less than one year, one, two, three, four, five, six, seven, eight, nine, ten, fifteen, twenty, or more years. In certain aspects, the relevant time for assessing prognosis or disease-free survival time may begin at the time of the surgical removal of the tumor or suppression, mitigation, or inhibition of tumor growth. A “good prognosis” refers to the likelihood that a cancer patient will survive for a period of at least five, such as for a period of at least ten years. In further aspects of the invention, a “poor prognosis” refers to the likelihood that a cancer patient, such as a melanoma patient, will experience disease relapse, tumor recurrence, metastasis, or death within less than ten years, such as less than five years or less than 1.5 years. Time frames for assessing prognosis and outcome provided herein are illustrative and are not intended to be limiting.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: FOXC2 expression is necessary to maintain the mesenchymal and invasive properties induced by EMT in mammary epithelial cells. FIG. 1A: Phase-contrast images of HMLE-Vector, -Snail, -Twist, and -TGF-β1 cells expressing either control-shRNA (shCntrl) or FOXC2-shRNA (shFOXC2). Scale bar indicates 100 μm. FIG. 1B: Expression of EMT marker mRNA by quantitative RT-PCR. GAPDH was used as an internal control. N=3; error bars indicate SEM. N.D.=not detected. FIG. 1C: Western blot analysis of EMT marker protein expression upon FOXC2 suppression in HMLE-Snail, -Twist, and -TGF-β1 cells. FIG. 1D: Quantification of invasion in Matrigel Transwell chambers using HMLE-Snail-shCntrl and HMLE-Snail-shFOXC2 cells in response to basic fibroblast growth factor (bFGF) and platelet-derived growth factor-BB (PDGF-BB). N=3; error bars indicate SEM. *P<0.05. FIG. 1E: Confocal microscopy images of HMLE-Snail-shCntrl and HMLE-Snail-shFOXC2 cells in 3D 1rECM cultures. The integrity of the basement membrane was assessed using anti-Laminin V with DAPI nuclear stain. Scale bar indicates 100 μm.

FIG. 2: FOXC2 expression is required for EMT-derived stem cell properties in mammary epithelial cells. FIG. 2A: Quantification of CD44 (CD44-PE) and CD24 (CD24-FITC) expression by FACS analysis in HMLE-Snail, -Twist, and -TGF-β1 cells expressing either shCntrl or shFOXC2. N=3; error bars indicated SEM. *P<0.05. FIG. 2B: In vitro quantification of mammospheres formed by cells described in FIG. 2A. N=3, error bars indicate SEM. *P<0.05. FIG. 2C: Quantification of cell viability using an MTS assay in cells described in FIG. 2A following culture for 96 hours in increasing concentrations of Paclitaxel. Data is represented as the absorbance (O.D.) at 490 nm. Top lines represent shCntrl; bottom lines represent shFOXC2. N=3, P<0.005.

FIG. 3: FOXC2 expression is increased in stem cell enriched populations and is sufficient to promote phenotypes associated with CSCs. FIG. 3A: Western blot analysis of FOXC2 expression in the stem cell enriched CD44^{hi}/CD24^{lo} (44^{hi}/24^{lo} and more differentiated CD44^{lo}/CD24^{hi} (44^{lo}/24^{hi}) cellular fractions isolated by FACS from HMLER and SUM159 cell lines. FIG. 3B: Western blot analysis of FOXC2 expression in cells cultured in monolayer culture (2D) and stem cell enriched mammosphere cultures (MS) for the indicated breast cancer cell lines. FIG. 3C: FACS analysis of CD44 and CD24 expression in HMLER-Vector and HMLER-FOXC2 cells. Representative FACS plots are shown. FIG. 3D: In vitro quantification of mammospheres formed by 1000 cells described in FIG. 3C. N=3; error bars indicate SEM. *P<0.05. FIG. 3E: Quantification of cell viability by MTS assay using HMLER cells expressing either Vector or FOXC2 cDNA following culture for 96 hours in increasing concentrations of Paclitaxel. Data represented is the mean absorbance (O.D.) at 490 nm. Top lines represent FOXC2; bottom lines represent Vector. N=3. FIG. 3F: Tumor incidence of FOXC2 expressing HMLER cells injected into the mammary fat pad of NOD/SCID mice in limiting dilutions. FIG. 3G: Tumor growth quantification of luciferase-labeled HMLER-Vector and HMLER-FOXC2 xenografts in vivo using bioluminescence after 28 days of inoculation into the mammary fat pad of NOD/SCID mice. N=5. FIG. 3H: Ex vivo bioluminescence images of the indicated organs of mice carrying HMLER-Vector and HMLER-FOXC2 xenografts after 28 days.

FIG. 4: FOXC2 derived gene signature is enriched in claudin-low human breast cancer samples and can accurately predict claudin-low human tumors. FIGS. 4A and B: Measurement of FOXC2 gene expression signature (GES) in MDA-MB231 (GSE12237) (FIG. 4A) and CN34 (GSE12237) (FIG. 4B) xenograft models consisting of the parental tumors and brain metastases. The box plots show the mean and 5% and 95% distributions of the level of FOXC2 signature (42) in GSE12237. Predicted activation of FOXC2 between the primary tumors and metastases was compared and a P-value was calculated using a Student's t-test. FIGS. 4C and D: The FOXC2 GES was scored in tumors (GSE18229) (FIG. 4C) and established breast cancer cell lines (E-TABM-157) (FIG. 4D). The box plots represent the mean and 5% and 95% distributions of the FOXC2 signature scores across the breast tumor subtypes data derived from GSE18229 (FIG. 4C) (35) and an expression dataset of 51 breast cancer cell lines described in (43) (FIG. 4D) (E-TABM-157; ArrayExpress). The one-way ANOVA significance for each plot was P<0.0001. FIG. 4E: Western blot analysis of FOXC2 expression in a panel of established breast cancer cell lines representing luminal, basal, and claudin-low subtypes.

FIG. 5: Attenuation of FOXC2 expression reduces the mesenchymal and stem cell properties of breast cancer cell lines with a claudin-low phenotype. FIG. 5A: Phase-contrast images of SUM159 and HMLER-Snail cells expressing either a control-shRNA (shCntrl) or FOXC2-shRNA (shFOXC2). Scale bar indicates 100 μm. FIG. 5B: Western blot analysis of EMT marker protein expression upon FOXC2 suppression in SUM159 and HMLER-Snail cells. FIGS. 5C and D: Quantification of Transwell cell migration for SUM159 (FIG. 5C) and HMLER-Snail (FIG. 5D) cells expressing either a control-shRNA (shCntrl) or FOXC2-shRNA (shFOXC2) in response to epidermal growth factor (EGF). Columns represent the average cell migration (N=6) relative to that induced in shCntrl cells by serum-free media (SFM) alone. Error bars indicate SEM. *P<0.05. FIG. 5E: Confocal microscopy images of SUM159-shCntrl and SUM159-shFOXC2 cells in 3D 1rECM cultures. Cell invasion was qualitatively assessed using anti-vimentin and F-actin detected with TRITC-conjugated phalloidin. Nuclei were stained with DAPI. Scale bar indicates 250 μm. FIG. 5F: Quantification of FACS analysis of CD44 (CD44-PE) and CD24 (CD24-FITC) expression in SUM159, MDA-MB-231, and HMLER-Snail cells expressing either shCntrl or shFOXC2. N=3; error bars indicated SEM. *P<0.05. FIG. 5G: In vitro quantification of mammospheres formed by 1000 cells described in FIG. 5F. N=3; error bars indicate SEM. *P<0.05. FIG. 5H: Tumor incidence of SUM159 and HMLER-Snail cells expressing shCntrl or shFOXC2 injected into the mammary fat pad of NOD/SCID mice in limiting dilutions and measured as palpable tumors after 12 weeks.

FIG. 6: FOXC2 regulates the expression of PDGFR-β. FIG. 6A: Western blot analysis of PDGFR-β expression in normal (i) and transformed (ii) mammary epithelial cells following EMT induction by multiple factors as well as in stem cell enriched)(44^{hi}/24^{lo} relative to (44^{lo}/24^{hi}) (iii) and mammosphere cultures (iv). FIG. 6B: Western blot analysis of PDGFR-β expression in a panel of established breast cancer cell lines representing luminal, basal, and claudin-low subtypes. FIG. 6C: Quantification of Transwell cell migration for the indicated cell lines in response to the PDGFR-β ligand, platelet-derived growth factor-BB (PDGF-BB) (20 ng/ml). N=6; error bars indicate SEM, *P<0.05. FIG. 6D: Phase contrast images of HMLER-Vector and HMLER-FOXC2 cells in 3D 1rECM cultures in the presence and absence of PDGF-BB. Scale bar indicates 50 μm. FIGS. 6E and F: Western blot analysis of PDGFR-β expression in cells induced to undergo EMT by ectopic expression of Twist, Snail, or TGF-β1 in HMLE derived cells (FIG. 6E) as well as in transformed cell lines (SUM159 and HMLER-Snail) (FIG. 6F) expressing either shCntrl or shFOXC2. FIG. 6G: Quantification of Transwell cell migration for the indicated cell lines expressing either a control-shRNA (shCntrl) or FOXC2-shRNA (shFOXC2) in response to PDGF-BB (20 ng/ml). N=6; error bars indicate SEM. *P<0.05. FIG. 6H: Quantification of binding of FOXC2 to the PDGFR-β promoter by ChIP assays. N=3; error bars represent the SEM. Capitalized nucleotides indicate the predicted FOXC2 binding sites at the indicated chromosomal locations.

FIG. 7: Sunitinib inhibits the growth and metastasis of FOXC2-expressing tumors. FIG. 7A: Quantification of HMLER-Vector and HMLER-FOXC2 cell viability in the presence of sunitinib (10 μM) relative to vehicle (DMSO). N=3; error bars indicate SEM. *P<0.05. FIG. 7B: In vitro quantification of mammospheres formed by 1000 HMLER-FOXC2 and HMLER-Snail cells in the presence of sunitinib (10 μM) or DMSO. N=3; error bars indicate SEM. *P<0.05. FIG. 7C: Tumor volume of HMLER-FOXC2 cells injected into the mammary fat pad of NOD/SCID mice and treated with 40 mg/kg of sunitinib or vehicle (N=9) daily for the indicated number of days. Top line represents Vehicle; bottom line represents Sunitinib. FIG. 7D: Event-free survival of mice with orthotopic HMLER-FOXC2 xenografts treated daily with sunitinib (40 mg/kg, N=7) or vehicle (N=10). Mice were euthanized once tumors reached 1.5 cm³. Bottom line represents Vehicle; top line represents Sunitinib. FIGS. 7E and F: Thirty days following initiation of sunitinib or vehicle treatment, mice were euthanized and the organs, lung (FIG. 7E) and brain (FIG. 7F), were dissected and analyzed for metastatic tumor burden using bioluminescence imaging. The luminescent signal of tumor cells is represented as the total photon flux detected in each organ from individual mice with the bar indicating the average. ***P<0.001, **P<0.05 compared to the vehicle control group.

FIG. 8: Wound healing assay with HMLE cells at 0 h and 9 h. The arrow indicates the co-expression of both FOXC2 and p-p38. Confluent cultures of HMLE cells were scratched with a pipet tip to make a wound, and the images were captured and stained for FOXC2 using immunofluorescence method at the beginning and at 9 h to observe the migration of the cells as well as expression of FOXC2 at the wound site.

FIG. 9: FOXC2 does not regulate the expression of other EMT factors. Quantification of EMT marker mRNA expression by quantitative real-time PCR analysis. Data is presented as the expression of EMT markers in HMLER-FOXC2 (FOXC2) cells relative to HMLER control cells. GAPDH was used as an internal normalization control. Columns indicate the mean (N=3); error bars indicate SEM.

FIG. 10: Ectopic expression of FOXC2 induces EMT in transformed human mammary epithelial cells (HMLER). FIG. 10A: Quantification of EMT marker mRNA expression by quantitative real-time PCR analysis. Data is represented as the expression of EMT markers in HMLER-FOXC2 (FOXC2) cells relative to HMLER-Vector control cells (Vector). GAPDH was used as an internal normalization control. Columns indicate the mean (N=3); error bars indicate SEM. FIG. 10B: Western blot analysis of EMT marker protein expression upon FOXC2 overexpression in HMLER cells. Actin was used as a loading control. FIG. 10C: Phase contrast and immunofluorescence images of HMLER-Vector and HMLER-FOXC2 cells. Overlaid images are shown for respective EMT markers and DAPI nuclear stain. Scale bar indicates 50 μm.

FIG. 11: Attenuation of FOXC2 expression leads to reduced levels of PDGFR-β. FIG. 11A: Immunofluorescence staining for PDGFR-β detected with Alexa Fluor 548 anti-mouse secondary antibody in HMLE, HMLE-Snail-shCntrl, and HMLE-Snail-shFOXC2 cells. Nuclei were counterstained with DAPI and overlaid images are shown. Scale bar indicates 100 μm. FIG. 11B: FACS analysis of PDGFR-β cell surface expression using anti-PDGFR-β-PE antibody in cells described in FIG. 11A. The histogram represents the intensity of PE signal in the FL2 detector on the x-axis. The curves on the histogram represent, from left to right, isotype control, parental HMLE, HMEL-Snail-shFOXC2, and HMLE-Snail-shCntrl.

FIG. 12: Cancer cells expressing endogenous FOXC2 exhibit increased sensitivity to PDGFR-β inhibitors such as sunitinib. FIGS. 12A and B: Quantification of HMLE-Snail (FIG. 12A) and SUM159 (FIG. 12B) cell viability expressing either shControl or shFOXC2, following culture for 96 hours in increasing concentrations of sunitinib. Cell viability was assessed using an MTS assay. Top lines represent shFOXC2; bottom lines represent shControl. FIG. 12C: In vitro quantification of mammospheres formed by cells described above grown in the presence of sunitinib. Data represents the number of mammospheres formed per 1000 cells seeded after 7 days of culture. For each cell type, the left column represents Vehicle and the right column represents Sunitinib. Error bars indicate SEM. *P<0.05.

### DESCRIPTION OF ILLUSTRATIVE EMBODIMENTS

Resistance to chemotherapy and metastases are the major causes of breast cancer-related mortality. Moreover, cancer stem cells (CSCs) play critical roles in cancer progression and treatment resistance. Previously, it was found that CSC-like cells can be generated by aberrant activation of EMT, thereby making anti-EMT strategies a novel therapeutic option for treatment of aggressive breast cancers. The inventors herein disclose that the transcription factor FOXC2 is induced in response to multiple EMT signaling pathways, is elevated in stem cell-enriched fractions, and is a critical determinant of mesenchymal and stem cell properties in cells induced to undergo EMT and CSC-enriched breast cancer cell lines. More specifically, attenuation of FOXC2 expression using lentiviral short hairpin RNA led to inhibition of the mesenchymal phenotype and associated invasive and stem cell properties, which included reduced mammosphere forming ability and tumor initiation. Overexpression of FOXC2 was sufficient to induce CSC properties and spontaneous metastasis in transformed human mammary epithelial cells. Furthermore, a FOXC2-induced gene expression signature was enriched in the claudin-low/basal B breast tumor subtype that contains EMT and CSC features. Having identified PDGFR-β to be regulated by FOXC2, the inventors demonstrate that the FDA-approved PDGFR inhibitor, sunitinib, targets FOXC2-expressing tumor cells leading to reduced CSC and metastatic properties. Thus, FOXC2 or its associated gene expression program may provide an effective marker and target for anti-EMT based therapies for the treatment of claudin-low/basal B breast tumors or other EMT/CSC-enriched tumors.

Since cells undergoing EMT are known to possess stem cell properties (Mani et al., 2008), and several recent studies independently demonstrated that CSCs as well as cells that have undergone EMT are relatively resistant to conventional chemo- and radio-therapies (Creighton et al., 2009; Li et al., 2008; Hollier et al., 2009; Gupta et al., 2009; Fillmore and Kuperwasser, 2008; Yu et al., 2007; Woodward et al., 2007), the EMT program may provide a novel therapeutic window for inhibiting CSCs. Due to the plethora of factors capable of inducing EMT and the hierarchy of the EMT programs, the inventors sought to identify a central functional mediator of EMT independent of the initiating signal that may provide a novel target for anti-EMT based therapies and found that FOXC2 serves as such a mediator. FOXC2 is induced by multiple factors and the expression of PDGFR-β is dependent on the expression of FOXC2. Therefore, PDGFR-β serves as a cells surface marker of CSCs, as well as cells that have undergone EMT, and a potential therapeutic target for cells that have undergone EMT. As such, sunitinib, a small molecule inhibitor capable of inhibiting PDGFR-β is capable of inhibiting CSCs in vitro as well as in vivo.

The present invention also relates to methods for determining that a cancer therapy or regimen is effective at targeting and/or impairing cancer stem cells by virtue of monitoring cancer stem cells over time and detecting a stabilization or decrease in the amount of cancer stem cells during and/or following the course of the cancer therapy or regimen.

### I. CANCER STEM CELLS

As used in the specification and claims, the terms “cancer stem cell(s)” and “CSC” are interchangeable and refer to solid cancer stem cells. CSCs are mammalian, and in preferred embodiments, these CSC are of human origin, but they are not intended to be limited thereto.

One hypothesis to explain how tumors grow and metastasize is the cancer stem cell hypothesis, which states that there is a small, distinct subset of cells within each tumor that is capable of indefinite self-renewal and of developing into the more adult tumor cell(s), which are relatively limited in replication capacity. It has been hypothesized that these cancer stem cells (CSC) might be more resistant to chemotherapeutic agents, radiation or other toxic conditions, and thus, persist after clinical therapies and later grow into secondary tumors, metastases or be responsible for relapse.

Solid tumors are thought to arise in organs that contain stem cell populations. The tumors in these tissues consist of heterogeneous populations of cancer cells that differ markedly in their ability to proliferate and form new tumors; this difference in tumor-forming ability has been reported for example with breast cancer cells and with central nervous system tumors. While the majority of the cancer cells have a limited ability to divide, recent literature suggests that a population of cancer cells, termed cancer stem cells, has the exclusive ability to extensively self-renew and form new tumors. Growing evidence suggests that pathways that regulate the self-renewal of normal stem cells are deregulated or altered in cancer stem cells, resulting in the continuous expansion of self-renewing cancer cells and tumor formation.

Cancer stem cells comprise a unique subpopulation (often 0.1%-10% or so) of a tumor that, relative to the remaining 90% or so of the tumor (i.e., the tumor bulk), are more tumorigenic, relatively more slow-growing or quiescent, and often relatively more chemoresistant than the tumor bulk. Given that conventional therapies and regimens have, in large part, been designed to attack rapidly proliferating cells (i.e. those cancer cells that comprise the tumor bulk), cancer stem cells, which are often slow-growing, may be relatively more resistant than faster growing tumor bulk to conventional therapies and regimens. Cancer stem cells can express other features that make them relatively chemoresistant, such as multi-drug resistance and anti-apoptotic pathways. The aforementioned would constitute a key reason for the failure of standard oncology treatment regimens to ensure long-term benefit in most patients with advanced stage cancers—i.e. the failure to adequately target and eradicate cancer stem cells. In some instances, a cancer stem cell(s) is the founder cell of a tumor (i.e., it is the progenitor of the cancer cells that comprise the tumor bulk).

A. Biomarkers

For the methods provided herein, the term biological samples refers to any biological sample obtained from an individual, including body fluids, body tissue, cells, or other sources known to those skilled in the art. Also, the terms “sample” and “biological sample” are used interchangeably herein. For example, a sample can be a tissue sample, such as a peripheral blood sample that contains circulating tumor cells, or a lung tumor tissue biopsy or resection. Other samples may include a thin layer cytological sample, a fine needle aspirate sample, a lung wash sample, a pleural effusion sample, a fresh frozen tissue sample, a paraffin embedded tissue sample, or an extract or processed sample produced from any of a peripheral blood sample. Body fluids, such as lymph, sera, whole fresh blood, peripheral blood mononuclear cells, frozen whole blood, plasma (including fresh or frozen), urine, saliva, semen, synovial fluid, and spinal fluid are also suitable as biological samples. Samples can further include breast tissue, renal tissue, colonic tissue, brain tissue, muscle tissue, synovial tissue, skin, hair follicle, bone marrow, and tumor tissue.

The biomarkers (also referred to herein as a “marker”) provided herein can be detected using any method known in the art.

1. FOXC2

The amino acid sequence and the cDNA sequence of human FOXC2, also called FREAC 11 or FKHL14, is described in International PCT Publication No. WO 98/54216, and in Miura et al. (1997), the teachings of which are hereby incorporated by reference in their entirety. In addition, the mouse form of FOXC2 is referred to as Fkh1/Mf1 in the art. Sequences of the mouse FOXC2 gene may be found in Miura et al. (1993). Additional sequences may be found in International PCT Publication WO01/60853, Genbank Accession No. NP_{—}005242 (Protein), and Genbank Accession No. NM_{—}005251 (mRNA sequence).

2. PDGFR-β

Platelet-derived growth factor receptors (PDGFRs) and their ligands, platelet-derived growth factors (PDGFs), play critical roles in mesenchymal cell migration and proliferation. The PDGFR/PDGF system includes two receptors (PDGFR-α and PDGFR-β) and four ligands (PDGF-A, B, C, and D). The receptors are plasma membrane-spanning proteins with intracellular tyrosine kinase domains. As with other protein kinases, activation of the PDGFRs is a key mechanism in regulating signals for cell proliferation, and abnormalities of PDGFR/PDGF are thought to contribute to a number of human diseases, such as atherosclerosis, balloon injury induced restenosis, pulmonary fibrosis, liver fibrosis, and especially malignancy.

The two receptors PDGFR-α and PDGFR-β are related in sequence (30% amino acid similarity), and have a common overall structure having four domains: an extracellular ligand binding domain consisting of five immunoglobulin-like structures, a transmembrane domain, a regulatory juxtamembrane domain and an intracellular catalytic domain. Both receptors are members of the class III subtype of receptor tyrosine kinases (RTKs), a group that shares a characteristic insertion sequence between two conserved elements of the tyrosine kinase domain. Normally, the receptors require dimerization (induced by binding of PDGF dimers) for autophosphorylation and activation. The PDGFR-β receptor strongly binds only the BB and DD dimers of PDGF, whereas the PDGFR-α receptor binds AA, BB and CC homodimers and AB heterodimers with similar affinity, but binds only weakly to DD homodimers.

In embryogenesis the PDGFR/PDGF system is essential for the correct development of the kidney, cardiovascular system, brain, lung and connective tissue. In adults, PDGFR/PDGF is important in wound healing, inflammation and angiogenesis. Human dermal fibroblasts appear to express seven times as much PDGFR-β receptor as PDGFR-α/β receptor, and the PDGFR-β receptor is responsible for most PDGF receptor phosphorylation

Agents that block PDGFR signaling have recently been found, such as ST1571 [also known as imatinib mesylate or Gleevec®].

B. Detection Methods

In certain embodiments, the method comprises the steps of obtaining a biological sample from a mammal to be tested; detecting the presence of a FOXC2 or PDGFR-β gene product in the sample, wherein if a FOXC2 or PDGFR-β gene product is present, then the mammal has an increased likelihood of having a tumor enriched with cancer stem cells. In one embodiment, the biological sample is a blood sample or a cell sample from a tumor in the mammal.

In one embodiment of the methods described herein, detecting the presence a gene product in a biological sample obtained from an individual comprises determining the level of an mRNA in the sample. The level of an mRNA in the sample can be assessed by combining oligonucleotide probes derived from the nucleotide sequence of the gene product to be detected with a nucleic acid sample from the individual, under conditions suitable for hybridization. Hybridization conditions can be selected such that the probes will hybridize only with the specified gene sequence. In one specific embodiment, conditions can be selected such that the probes will hybridize only with an altered nucleotide sequences, such as but not limited to, splice isoforms, and not with unaltered nucleotide sequences; that is, the probes can be designed to recognize only particular alterations in the nucleic acid sequence of the mRNA, including addition of one or more nucleotides, deletion of one or more nucleotides or change in one or more nucleotides (including substitution of a nucleotide for one which is normally present in the sequence). In one specific embodiment, the oligonucleotide probe hybridizes to the FOXC2 mRNA sequence set forth as Genbank Deposit No. NM_{—}005251, or to the coding region of the mRNA sequence.

Methods of quantitating mRNA in a sample are well-known in the art. In a particular embodiment, oligonucleotide probes specific to FOXC2 can be displayed on an oligonucleotide array or used on a DNA chip. The term “microarray” refers to an array of distinct polynucleotides or oligonucleotides synthesized on a substrate, such as paper, nylon or other type of membrane, filter, chip, glass slide, or any other suitable solid support. Microarrays also include protein microarrays, such as protein microarrays spotted with antibodies. Other techniques for detecting FOXC2 mRNA levels in a sample include reverse transcription of mRNA, followed by PCR amplification with primers specific for a FOXC2 mRNA (e.g., RT-PCR or quantitative RT-PCR), in situ hybridization, Northern blotting, or nuclease protection.

Quantitative real-time PCR (qRT-PCR) may also be used to measure the differential expression of a plurality of biomarkers. In qRT-PCR, the RNA template is generally reverse transcribed into cDNA, which is then amplified via a PCR reaction. The amount of PCR product is followed cycle-by-cycle in real time, which allows for determination of the initial concentrations of mRNA. To measure the amount of PCR product, the reaction may be performed in the presence of a fluorescent dye, such as SYBR Green, which binds to double-stranded DNA. The reaction may also be performed with a fluorescent reporter probe that is specific for the DNA being amplified.

A non-limiting example of a fluorescent reporter probe is a TaqMan® probe (Applied Biosystems, Foster City, Calif.). The fluorescent reporter probe fluoresces when the quencher is removed during the PCR extension cycle. Multiplex qRT-PCR may be performed by using multiple gene-specific reporter probes, each of which contains a different fluorophore. Fluorescence values are recorded during each cycle and represent the amount of product amplified to that point in the amplification reaction. To minimize errors and reduce any sample-to-sample variation, qRT-PCR may be performed using a reference standard. The ideal reference standard is expressed at a constant level among different tissues, and is unaffected by the experimental treatment.

Suitable reference standards include, but are not limited to, mRNAs for the housekeeping genes glyceraldehyde-3-phosphate-dehydrogenase (GAPDH) and β-actin. The level of mRNA in the original sample or the fold change in expression of each biomarker may be determined using calculations well known in the art.

In situ hybridization may also be used to measure the differential expression of a plurality of biomarkers. This method permits the localization of mRNAs of interest in the cells of a tissue section. For this method, the tissue may be frozen, or fixed and embedded, and then cut into thin sections, which are arrayed and affixed on a solid surface. The tissue sections are incubated with a labeled antisense probe that will hybridize with an mRNA of interest. The hybridization and washing steps are generally performed under highly stringent conditions. The probe may be labeled with a fluorophore or a small tag (such as biotin or digoxigenin) that may be detected by another protein or antibody, such that the labeled hybrid may be detected and visualized under a microscope. Multiple mRNAs may be detected simultaneously, provided each antisense probe has a distinguishable label. The hybridized tissue array is generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for each biomarker.

In another embodiment of the methods described herein, detecting the presence a gene product in a biological sample obtained from an individual comprises determining the level of a polypeptide in the sample. The level of a gene product can be determined by contacting the sample with an antibody that specifically binds to the polypeptide product and determining the amount of bound antibody, e.g., by detecting or measuring the formation of the complex between the antibody and the polypeptide. The antibodies can be labeled (e.g., radioactive, fluorescently, biotinylated or HRP-conjugated) to facilitate detection of the complex. Appropriate assay systems for detecting polypeptide levels include, but are not limited to, flow cytometry, Enzyme-Linked Immunosorbent Assay (ELISA), competition ELISA assays, Radioimmuno-Assays (RIA), immunofluorescence, gel electrophoresis, Western blot, and chemiluminescent assays, bioluminescent assays, immunohistochemical assays that involve assaying a gene product in a sample using antibodies having specificity for the polypeptide product. Numerous methods and devices are well known to the skilled artisan for the detection and analysis of the instant invention. With regard to polypeptides or proteins in test samples, immunoassay devices and methods are often used. These devices and methods can utilize labeled molecules in various sandwich, competitive, or non-competitive assay formats, to generate a signal that is related to the presence or amount of an analyte of interest. Additionally, certain methods and devices, such as but not limited to, biosensors and optical immunoassays, may be employed to determine the presence or amount of analytes without the need for a labeled molecule.

Alternatively, the level of FOXC2 polypeptide may be detected using mass spectrometric analysis. Mass spectrometric analysis has been used for the detection of proteins in serum samples. Mass spectroscopy methods include Surface Enhanced Laser Desorption Ionization (SELDI) mass spectrometry (MS), SELDI time-of-flight mass spectrometry (TOF-MS), Maldi Qq TOF, MS/MS, TOF-TOF, ESI-Q-TOF and ION-TRAP.

A polypeptide can be detected and quantified by any of a number of means known to those of skill in the art, including analytic biochemical methods, such as electrophoresis, capillary electrophoresis, high performance liquid chromatography (“HPLC”), thin layer chromatography (“TLC”), hyperdiffusion chromatography, and the like, or various immunological methods, such as fluid or gel precipitation reactions, immunodiffusion (single or double), immunoelectrophoresis, radioimmunoassay (“RIA”), enzyme-linked immunosorbent assay (“ELISA”), immunofluorescent assays, flow cytometry, FACS, western blotting, and the like.

Immunohistochemical staining may also be used to measure the differential expression of a plurality of biomarkers. This method enables the localization of a protein in the cells of a tissue section by interaction of the protein with a specific antibody. For this, the tissue may be fixed in formaldehyde or another suitable fixative, embedded in wax or plastic, and cut into thin sections (from about 0.1 mm to several mm thick) using a microtome. Alternatively, the tissue may be frozen and cut into thin sections using a cryostat. The sections of tissue may be arrayed onto and affixed to a solid surface (i.e., a tissue microarray). The sections of tissue are incubated with a primary antibody against the antigen of interest, followed by washes to remove the unbound antibodies. The primary antibody may be coupled to a detection system, or the primary antibody may be detected with a secondary antibody that is coupled to a detection system. The detection system may be a fluorophore or it may be an enzyme, such as horseradish peroxidase or alkaline phosphatase, which can convert a substrate into a colorimetric, fluorescent, or chemiluminescent product. The stained tissue sections are generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for the biomarker.

An enzyme-linked immunosorbent assay, or ELISA, may be used to measure the differential expression of a plurality of biomarkers. There are many variations of an ELISA assay. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art.

An antibody microarray may also be used to measure the differential expression of a plurality of biomarkers. For this, a plurality of antibodies is arrayed and covalently attached to the surface of the microarray or biochip. A protein extract containing the biomarker proteins of interest is generally labeled with a fluorescent dye.

The labeled biomarker proteins are incubated with the antibody microarray. After washes to remove the unbound proteins, the microarray is scanned. The raw fluorescent intensity data may be converted into expression values using means known in the art.

### II. TREATMENT OF NEOPLASTIC CONDITIONS

The term “patient” means all mammals including humans. Examples of patients include humans, cows, dogs, cats, goats, sheep, pigs, and rabbits. Preferably, the patient is a human.

A “disorder” or “disease” is any condition that would benefit from treatment with a substance/molecule or method of the invention. This includes chronic and acute disorders or diseases including those pathological conditions that predispose the mammal to the disorder in question. Furthermore, non-limiting examples of disorders to be treated herein include malignant and benign tumors; non-leukemias and lymphoid malignancies; neuronal, glial, astrocytal, hypothalamic, and other glandular, macrophagal, epithelial, stromal, and blastocoelic disorders; and inflammatory, immunologic, and other angiogenic disorders.

The methods described herein are useful in treating cancer, particularly, metastatic disease and after adjuvant therapy, such as surgery or radiotherapy. Generally, the terms “cancer” and “cancerous” refer to or describe the physiological condition in mammals that is typically characterized by unregulated cell growth. More specifically, cancers that are treated using any one or more tyrosine kinase inhibitors, other drugs blocking the receptors or their ligands, or variants thereof, and in connection with the methods provided herein include, but are not limited to, carcinoma, lymphoma, blastoma, sarcoma, leukemia, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer and gastrointestinal stromal cancer), pancreatic cancer, glioblastoma, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, melanoma, superficial spreading melanoma, lentigo maligna melanoma, acral lentiginous melanomas, nodular melanomas, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

An effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

Tyrosine kinases are a subgroup of the larger class of protein kinases. Fundamentally, a protein kinase is an enzyme that modifies a protein by chemically adding phosphate groups via phosphorylation. Such modification often results in a functional change to the target protein or substrate by changing the enzyme activity, cellular location, or association with other proteins. Chemically, the kinase removes a phosphate group from ATP and covalently attaches it to one of three amino acids (serine, threonine, or tyrosine) that have a free hydroxyl group. Most kinases act on both serine and threonine, and certain others, tyrosine. There are also a number of kinases that act on all three of these amino acids. Generally, kinases are enzymes known to regulate the majority of cellular pathways, especially pathways involved in signal transduction or the transmission of signals within a cell. Because protein kinases have profound effects on a cell, kinase activity is highly regulated. Kinases can be turned on or off by phosphorylation (sometimes by the kinase itself through cis-phosphorylation/autophosphorylation) and by binding to activator proteins, inhibitor proteins, or small molecules.

Treatment outcomes can be predicted and monitored and/or patients benefiting from such treatments can be identified or selected via the methods described herein for the PDGFR-β inhibitors. As such, PDGFR-β inhibitors useful in the present invention include, but are not limited to, sunitinib, axitinib, BIBF1120, MK-2461, dovitinib, pazopanib, telatinib, CP 673451, or TSU-68. A preferred inhibitor is sunitinib.

Administration in combination can include simultaneous administration of two or more agents in the same dosage form, simultaneous administration in separate dosage forms, and separate administration. That is, the subject therapeutic composition and another therapeutic agent can be formulated together in the same dosage form and administered simultaneously. Alternatively, subject therapeutic composition and another therapeutic agent can be simultaneously administered, wherein both the agents are present in separate formulations. In another alternative, the therapeutic agent can be administered just followed by the other therapeutic agent or vice versa. In the separate administration protocol, the subject therapeutic composition and another therapeutic agent may be administered a few minutes apart, or a few hours apart, or a few days apart.

Regarding neoplastic condition treatment, depending on the stage of the neoplastic condition, neoplastic condition treatment involves one or a combination of the following therapies: surgery to remove the neoplastic tissue, radiation therapy, and chemotherapy. Other therapeutic regimens may be combined with the administration of the anticancer agents, e.g., therapeutic compositions and chemotherapeutic agents. For example, the patient to be treated with such anti-cancer agents may also receive radiation therapy and/or may undergo surgery.

For the prevention or treatment of disease, the appropriate dosage of an therapeutic composition, e.g., a PDGFR-β inhibitor, will depend on the type of disease to be treated, as defined above, the severity and course of the disease, whether the agent is administered for preventive or therapeutic purposes, previous therapy, the patient's clinical history and response to the agent, and the discretion of the attending physician. The agent is suitably administered to the patient at one time or over a series of treatments.

A. Combination Treatments

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. A tissue, tumor, or cell can be contacted with one or more compositions or pharmacological formulation(s) comprising one or more of the agents or by contacting the tissue, tumor, and/or cell with two or more distinct compositions or formulations. Also, it is contemplated that such a combination therapy can be used in conjunction with radiotherapy, surgical therapy, or immunotherapy.

A PDGFR-β may be administered before, during, after, or in various combinations relative to an anti-cancer treatment. The administrations may be in intervals ranging from concurrently to minutes to days to weeks. In embodiments where the PDGFR-β inhibitor is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the PDGFR-β inhibitor and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below an antibody therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gamma1I and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the invention. In the context of cancer treatment, immunotherapeutics, generally, rely on the use of immune effector cells and molecules to target and destroy cancer cells. Rituximab (Rituxan®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present invention, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present invention to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present invention to improve the treatment efficacy.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### FOXC2 is Required for the Maintenance of the Mesenchymal Phenotype Following EMT Induction in Human Mammary Epithelial Cells

Enhanced expression of FOXC2 has been observed following the induction of EMT by several factors in experimentally immortalized human mammary epithelial (HMLE) cells (Mani et al., 2007) strongly suggesting that FOXC2 may be a critical determinant of multiple EMT programs. To assess the functional role of FOXC2 during EMT, shRNA mediated suppression of FOXC2 was employed in HMLE cells that underwent EMT via ectopic expression of Snail, Twist, or TGF-131. The suppression of FOXC2 had no significant effect on cell growth, but substantially altered the in vitro morphology of all cell lines, including increased clustering of cells into epithelial-like islands with prominent cell-cell contacts and reduced fibroblastic morphology (FIG. 1A). FOXC2 attenuation also led to reduced expression of mesenchymal markers vimentin, fibronectin, and N-cadherin across all cell types tested as well as re-expression (both mRNA and protein levels) of the epithelial marker E-cadherin in HMLE-Snail and HMLE-TGF-β1 cells (FIGS. 1B and C). To examine whether FOXC2 could function in parallel to other EMT regulators, the expression of Snail, Twist, and Slug was examined in cells ectopically expressing FOXC2 and found only moderate upregulation of Twist but not Snail or Slug expression (FIG. 9) suggesting FOXC2 may not regulate expression of these factors.

The passage of cells through EMT and the acquisition of mesenchymal properties is associated with increased migratory and invasive properties. Suppression of FOXC2 was observed to significantly inhibit the invasion of HMLE-Snail cells through Matrigel using Transwell migration assays in response to the soluble chemotactic ligands basic fibroblast growth factor (bFGF) and platelet-derived growth factor-BB (PDGF-BB) (FIG. 1D). Mammary cells possessing epithelial traits are known to form organized multicellular acini structures with an intact laminin positive basement membrane in 3D laminin-rich extracellular matrix Matrigel cultures (3D 1rECM) (Petersen et al., 1992; Muthuswamy et al., 2001). However, following the induction of EMT, cells became disorganized and gained invasive properties characterized by disrupted laminin staining (Kim et al., 2007; Viloria-Petit et al., 2009). Using 3D 1rECM assays, HMLE-Snail control-shRNA cells grew as highly invasive stellate structures with disrupted basement membrane, displayed by disorganized laminin V staining (FIG. 1E). In contrast, the HMLE-Snail FOXC2-shRNA cells formed non-invasive, multicellular structures with an intact basement membrane as depicted by a continuous laminin V layer (FIG. 1E). Taken together, these results suggest that FOXC2 is necessary for the maintenance of the mesenchymal phenotype of mammary epithelial cells following passage through EMT.

### Example 2

### FOXC2 is Necessary for the Stem Cell-Like Properties Generated Via EMT in Mammary Epithelial Cells

Previously, HMLE-Snail, -Twist and -TGF-β1 cells were known to aquire properties similar to breast CSCs, including the CD44^{high}/CD24^{low} cell surface markers and an increased ability to form mammospheres (Mani et al., 2008). Here, the attenuation of FOXC2 expression by shRNA in these cells was found to reduce the number of cells with the CD44^{high}/CD24^{low} phenotype compared to the control-shRNA expressing cells (FIG. 2A). In the same conditions, there was a marked decrease in the mammosphere forming ability (FIG. 2B).

Next, the acquisition of stem cell properties and passage through EMT has been reported to increase the resistance to chemotherapeutic agents (Hollier et al., 2009; Gupta et al., 2009). In accordance with this, suppression of FOXC2 expression in cells that have undergone EMT (HMLE-Snail, HMLE-Twist and HMLE-TGF-131) was found to sensitize them to paclitaxel (FIG. 2C). Together, these results indicate FOXC2 expression is necessary for EMT-derived stem cell-like properties in mammary epithelial cells.

### Example 3

### FOXC2 is Elevated in CSC-Enriched Populations and is Sufficient to Promote the Generation of CSCs and Metastatic Competence in Transformed Human Mammary Epithelial Cells

Previously, FACS isolation of breast tumor cells with the CD44^{high}/CD24^{low} cell surface phenotype (Al-Hajj et al., 2003) as well as isolation of tumor cells from mammospheres (Dontu et al., 2003) were demonstrated to be able to enrich for populations of tumor initiating CSCs. Using this rationale, the inventors FACS isolated CD44^{high}/CD24^{low} and CD44^{low}/CD24^{high} cellular fractions from HMLER and SUM159 breast cancer cell lines and observed increased FOXC2 protein expression in the CD44^{high}/CD24^{low} stem cell fraction relative to the CD44^{low}/CD24^{high} fraction (FIG. 3A). Increased FOXC2 protein expression was also observed in primary mammospheres isolated from HMLER, SUM159, HCC38, and SUM149 cells as compared to same cells grown in monolayer cultures (FIG. 3B).

Previously, ectopic expression of FOXC2 was found to induce partial EMT in Madin-Darby canine kidney (MDCK) epithelial cells, and was sufficient to promote the metastasis of EpRas murine mammary carcinoma cells (Mani et al., 2007). However, whether FOXC2 expression alone can induce EMT and generate CSC-like properties remains unknown. The inventors found that ectopic expression of FOXC2 in HMLER cells is sufficient to induce a robust EMT and subsequent CSC-like properties, both in vitro and in vivo. This was displayed as a characteristic switch of morphology to a fibroblastic appearance with a reduction in epithelial specific E-cadherin, in conjunction with increased expression of mesenchymal markers vimentin, fibronectin, and N-cadherin at the mRNA and protein levels (FIGS. 10A-C). Furthermore, HMLER-FOXC2 cells had increased CSC-like properties, including a switch to the CD44^{high}/CD24^{low} cell surface phenotype (FIG. 3C), enhanced mammosphere-forming efficiency (FIG. 3D), and increased resistance to paclitaxel (FIG. 3E). Notably, overexpression of FOXC2 led to increased tumor formation of HMLER cells in the mammary fat-pad (FIG. 3F). In fact, as few as 1×10³ HMLER-FOXC2 cells could robustly initiate tumors (7/8 sites), while at least 2×10⁶ HMLER-Vector control cells were required to get a 54% tumor take rate (7/13 sites) within the same 12 week time frame (FIG. 3F).

To assess the effect of FOXC2 expression on the orthotopic growth and spontaneous metastasis of HMLER cells, 2×10⁶ luciferase-labeled HMLER-FOXC2 or HMLER-Vector cells were injected into the mammary fat-pad of NOD/SCID mice and analyzed for metastases using bioluminescent imaging once per week until the tumors either reached a volume of 1.5 cm³ (28 days for HMLER-FOXC2) or until the end of the experiment after 12 weeks (HMLER-Vector). FOXC2 expression promoted aggressive growth (FIG. 3G) and metastasis of HMLER cells to the lungs, liver, hind leg bone, and most strikingly, to the brain (FIG. 3H) as soon as 28 days post-injection. In contrast HMLER-Vector cells did not metastasize to any of the organs examined (FIG. 3H) even at the end of the experiment. Collectively, these findings suggest that FOXC2 expression is sufficient to promote EMT and CSC properties, including chemoresistance, tumor initiation, and metastatic competence, in transformed human mammary epithelial cells.

### Example 4

### A FOXC2-Associated Gene Expression Signature is Enriched in Claudin-Low Human Breast Tumors

The inventors investigated whether FOXC2 activity could be observed in metastatic tumors in vivo. As a measure of FOXC2 activity, the inventors generated, using microarray data, a FOXC2 gene expression signature (GES) by comparing the gene expression profile of HMLER cells overexpressing FOXC2 to vector-transduced counterparts. Applying this GES to data from two human xenograft models, MDA-MB-231 and CN34, it was observed that the FOXC2 GES was significantly higher in metastases compared with the primary tumors in both datasets (FIGS. 4A and B). Collectively, these data indicate that the FOXC2-associated signature is enriched in metastases relative to primary tumors.

Previously, elevated FOXC2 expression was found to correlate with basal-like breast cancers (Mani et al., 2007). However, using gene expression profiling, an aggressive claudin-low group has been found within this subtype (Herschkowitz et al., 2007; Prat et al., 2010). The FOXC2 GES was found to be significantly enriched in claudin-low human breast tumors (FIG. 4C), as well as in claudin-low cell lines (FIG. 4D) compared to other subtypes. To verify this finding, a western blot analysis was performed and it was found that all claudin-low cell lines analyzed (6/6) expressed FOXC2 at varying degrees, while none of the other cell lines expressed significant levels of FOXC2 (FIG. 4E).

### Example 5

### FOXC2 is Required for the Mesenchymal and CSC-Like Properties of Claudin-Low Breast Cancer Cells

As FOXC2 expression and activity was found to be associated with claudin-low breast tumors, it was tested whether FOXC2 expression was required for the mesenchymal and invasive properties of SUM159, MDA-MB231, and HMLER-Snail cells that have a claudin-low gene expression phenotype (Prat et al., 2010). The suppression of FOXC2 resulted in a less fibroblastic morphology with increased epithelial-like cell clustering (FIG. 5A), decreased expression of mesenchymal markers fibronectin and N-cadherin (FIG. 5B) and re-expression of E-cadherin in HMLER-Snail cells (FIG. 5B). Furthermore, FOXC2 suppression significantly reduced Transwell migration of both SUM159 and HMLER-Snail cells (FIGS. 5C and D) (p<0.05). In a similar fashion, SUM159 Control-shRNA cells grew in 3D 1rECM cultures with a characteristic stellate morphology (Kenny et al., 2007) and extended multicellular protrusions invading into the surrounding Matrigel (FIG. 5E). The suppression of FOXC2 resulted in a dramatic reduction in the invasive morphology of SUM159 cells (SUM159 FOXC2-shRNA) compared to control cells (FIG. 5E).

Next, the effect of FOXC2 suppression on stem cell properties in the same three cell lines was assessed. Suppression of FOXC2 expression substantially reduced the percentage of cells displaying the CD44^{high}/CD24^{low} CSC-like cell surface profile (FIG. 5F) as well as significantly abrogating the mammosphere forming ability (FIG. 5G) as compared to the Control-shRNA expressing cells. Given that suppression of FOXC2 expression diminished stem cell properties in vitro, the inventors next examined if FOXC2 knockdown affected the tumor-initiating potential of SUM159 as well as HMLER-Snail cells using limiting dilution tumor-initiation assays. FOXC2-shRNA or Control-shRNA expressing cells were introduced into the mammary fat-pad of NOD/SCID mice, and it was found that the suppression of FOXC2 expression decreased tumor initiation frequency relative to control cells of both SUM159 and HMLER-Snail xenografts (FIG. 5H). In summary, these findings suggest that FOXC2 may be an important functional mediator of the mesenchymal and CSC properties of claudin-low breast cancer cells.

### Example 6

### FOXC2 Regulates PDGFR-β Expression

Cells induced to undergo EMT by multiple factors were found to upregulate the expression of PDGFR-β (CD140b) (Battula et al., 2010) similar to FOXC2. Thus, it was hypothesized that PDGFR-β might provide a druggable downstream target in FOXC2 expressing cells. First, it was confirmed that PDGFR-β protein expression was upregulated in a panel of EMT-derived cells (FIGS. 6Ai and ii). As observed for FOXC2 (FIGS. 3A and B), the expression of PDGFR-β was elevated in the stem cell-enriched CD44^{high}/CD24^{low} subpopulation of HMLER cells as well as in SUM159 cells cultured as mammospheres, compared to controls (FIGS. 6A iii and iv). Furthermore, the expression pattern of PDGFR-β and FOXC2 correlated strongly across a panel of established cell lines with increased expression observed in claudin-low cell lines SUM159 and Hs578T (FIG. 6B). Reflecting the increased levels of PDGFR-β in EMT-derived and claudin-low cell lines, the addition of the ligand PDGF-BB significantly elevated Transwell cell migration in all cells tested (FIG. 6C) and led to the further enhancement of the invasive phenotype of HMLER-FOXC2 cells in 3D 1rECM cultures (FIG. 6D).

Next, the inventors investigated if PDGFR-β expression is dependent on FOXC2 expression. Across a panel of cells, the expression of PDGFR-β was found to be substantially decreased upon suppression of FOXC2 (FIGS. 6E, 6F, and 11). Consequently, the suppression of FOXC2 in HMLE-Snail, HMLE-TGF-β1, HMLER-Snail, and SUM159 cells compromised the ability of these cells to migrate towards PDGF-BB (FIG. 6G). To examine the possibility of FOXC2 directly regulating PDGFR-β transcription, the inventors performed a chromatin immunoprecipation (ChiP) assay using HMLER-FOXC2 cells. FOXC2 preferentially bound to two regions at 2.7 kb (−2.7 kb) and 1.3 kb (−1.3 kb) upstream of the PDGFR-β transcription start site (FIG. 6H), thus demonstrating that FOXC2 may be a direct transcriptional regulator of PDGFR-β expression.

### Example 7

### Sunitinib Targets FOXC2-Expressing Tumors

Since it was found that expression of PDGFR-β is regulated by FOXC2, whether sunitinib, a small molecule inhibitor of PDGFR-β, could suppress the stem-like and metastatic properties of FOXC2-expressing cells was tested. Sunitinib treatment was found to specifically inhibit the cell growth of HMLER-FOXC2 but not HMLER-Vector control cells in monolayer cultures (FIG. 7A). The treatment of HMLER-FOXC2 and HMLER-Snail cells with sunitinib was found to significantly decrease mammosphere formation by >8-fold and >20-fold, respectively (FIG. 7B), as compared to vehicle (DMSO) treated cells. Furthermore, cells expressing endogenous FOXC2 also display increased sensitivity to sunitinib as evidenced by both MTS assay and reduction in mammosphere formation (FIG. 4).

To investigate whether sunitinib could inhibit FOXC2-expressing tumors in vivo, sunitinib was orally administered to mice following orthotopic injection of luciferase labeled HMLER-FOXC2 cells into the mammary fat-pad. In accordance with the in vitro observations, sunitinib treatment reduced primary tumor growth (FIG. 7C) and extended event-free survival of mice carrying FOXC2 tumors compared to vehicle-treated control mice (FIG. 7D). Also, the lungs (FIG. 7E) and brain (FIG. 7F) of sunitinib-treated mice were analyzed for the presence of HMLER-FOXC2 metastases and it was found that the sunitinib-treated group had significantly reduced metastatic burden compared to the vehicle-treated mice as evidenced by significantly lower photon counts in these organs following dissection (FIGS. 7E and F). Taken together, these results indicate that FOXC2-expressing tumor cells are sensitive to PDGFR-targeted therapies and suggest that sunitinib may be an effective means of targeting FOXC2-expressing cell populations, which may include EMT-derived CSC-like and metastatic phenotypes.

### Example 8

### Wound Healing for In Vitro EMT Assay, Monitoring FOXC2 Induction and Screening for Drugs, which could Inhibit FOXC2 and the Associated EMT/CSC Properties

Metastasis is a multistep process involving the dissemination of cancer cells from the primary tumors, survival in the circulation, and establishment of secondary tumors. The epithelial-to-mesenchymal transition (EMT) plays an important role in tumor progression. The characteristic hallmarks of EMT include loss of cell-cell adhesion and lack of basal lamina. Recently, it was found that the cancer cells acquire stem cell properties during EMT to complete the complex cascade of metastatic events. In addition, it was found that the FOXC2 transcription factor is preferentially induced and expressed in cells having undergone EMT and that FOXC2 is an important functional mediator of both mesenchymal and stem cell properties in cells having undergone EMT.

Similar to metastasis, the EMT process is also known to facilitate wound healing. For example, when a wound is induced in cells cultured in vitro, the cells at the damaged edges migrate toward the center of the wound, and cover and heal the wound surface. It has been known that the cells at the edge acquire EMT properties, such as motility, and are thereby able to migrate to heal the inflicted wound. Since FOXC2 is induced following EMT, as well as in cancer stem cells, the inventors investigated the activation/induction of FOXC2 at the wound site.

A striking upregulation/induction of FOXC2 was found only at the wound site and not away from the wound relative to the 0 hour time point (FIG. 8). Since FOXC2 can be induced dynamically in vitro, this assay may be used to screen for drugs that inhibit EMT/CSC properties and will indirectly provide an important screening tool for drugs targeting cancer metastases, therapy resistant tumors, as well as tumor relapse.

### Example 9

### Materials and Methods

Cell Lines, Culture Conditions, and Antibodies.

Immortalized human mammary epithelial cells (HMLE) and V12H-Ras transformed derivatives (HMLER), including cells expressing empty vector (pWZL), Snail, Twist, Goosecoid (GSC), or an activated form of TGF-β1 were maintained as previously described (8). Established human breast cancer cell lines were cultured in cell specific medium (Table 1). Antibodies used included anti-β-actin (Abcam), FOXC2 (Dr. Naoyuki Miura), E-cadherin (BD Bioscience), Fibronectin (BD Bioscience), N-cadherin (BD Bioscience), Vimentin (NeoMarkers), and β-catenin (BD Bioscience).

TABLE 1
Cell type specific media used for breast cancer cell lines.

| Cell Line | Basal Media | Additives* |
|---|---|---|
| MCF-7 | DMEM/F12 | 10% FBS |
| ZR75B | DMEM/F12 | 10% FBS |
| MDA-MB-468 | RPMI 1640 | 5% FBS |
| MDA-MB-231 | RPMI 1640 | 5% FBS |
| BT-20 | RPMI 1640 | 10% FBS |
| HCC38 | RPMI 1640 | 10% FBS |
| Hs578T | DMEM | 10% FBS |
| SUM149 | F12 | 5% FBS/Ins/Hyd |
| SUM159 | F12 | 5% FBS/Ins/Hyd |
| MCF10A | DMEM/F12 | 5% Horse serum/Ins/Hyd cholera toxin |
| MDA-MB-436 | DMEM | 10% FBS |
| T47D | RPMI 1640 | 10% FBS |
| BT549 | RPMI 1640 | 10% FBS |
| BT474 | RPMI 1640 | 10% FBS |
| MDA-MB-435 | DMEM | 10% FBS |

*All media contains penicillin (50 units/ml) and streptomycin (0.1 μg/ml).
Ins = Insulin; Hyd = Hydrocortisone.

For assays characterizing the resistance of cells to cytotoxic compounds, cells were seeded at a density of 8×10³ cells per well in 96-well plates. After 24 hours, medium was replaced with fresh growth medium (100 μl/well) containing the indicated concentrations of Paclitaxel (Sigma) dissolved in DMSO. Cell viability was assessed after 96 hours using the CellTiter96 Aqueous One Solution Cell Proliferation Assay (Promega).

To determine the effects of sunitinib on cell growth, 2×10⁵ cells were plated in a 6-well tissues culture dish. After 24 hours, the media was replaced with growth media containing 2.5 μM sunitinib, and the media was replace every 2 days. On the sixth day of treatment, viable cells per well was quantified using a Beckman-Coulter Vi-Cell Viability Analyzer.

Plasmids and Viral Transduction.

The production of lentiviruses and amphotrophic retroviruses, and the transduction of target cells were performed as previously described (Stewart et al., 2003). HMLE derived cell lines, HMLE-Snail, HMLE-Twist, HMLE-GSC, and HMLE-TGF-β1, were generated using retroviral transduction of cells using the pWZL-Blast construct encoding the relevant cDNA and selection in 4 μg/ml of blasticidin (Invitrogen). HMLER-FOXC2 and HMLER-Snail cells were generated in two steps, via which 1) HMLER cells were first generated by the transformation of HMLE cells by infection with the retroviral MSCV-H-RasV12-IRES-GFP vector (Addgene plasmid #18780) and FACS isolation of GFP-positive cells after 2 weeks of culture; and 2) stable expression of human FOXC2 and Snail cDNA following infection of HMLER cells with pWZL-Blast-FOXC2 and pWZL-Blast-Snail vectors and selection with 4 μg/ml blasticidin to generate HMLER-FOXC2 and HMLER-Snail cell lines, respectively. To suppress FOXC2 expression, the shRNA-expressing pLKO lentivirus system was used (OpenBiosystems). The FOXC2 shRNA targeting sequences were CCTGAGCGAGCAGAATTACTA (pLKO5; SEQ ID NO: 15) and GCGGGAGATGTTCAACTCCCA (pLKO4; SEQ ID NO: 16). The shRNA sequences targeting firefly luciferase (shCntrl) or GFP in the pLKO vector were used as controls. The stable suppression of target genes was achieved by selection of cells in 2 μg/ml of puromycin.

Mammosphere Assay.

Mammosphere cultures were performed as described (Dontu et al., 2003), with the exception that culture medium contained 1% methylcellulose to reduce cell aggregation (Mani et al., 2008). One thousand cells were plated per well into 96-well plates and cultured for 7-10 days, with fresh medium replaced every 3 days. Following incubation, mammospheres were photographed and spheres with a diameter greater than 75 μm were counted. To test the effects of sunitinib, 1000 cells were plated in 96-well low attachment plate in 100 μl mammosphere media. At 24 hours and 96 hours, 100 μl of mammosphere media containing 10 μM sunitinib or vehicle control (DMSO) was added to each well. Spheres were quantified after 7 days.

Three Dimensional (3D) Laminin-Rich Extracellular Matrix (1rECM) On-Top Cultures.

The 3D 1rECM on-top cultures were adapted from the procedures previously described (Lee et al., 2007). Cells in culture were trypsinized and seeded at a density of 2.5×10³ cells per well on top of a thin gel of Engelbreth-Holm-Swarm tumor extract (Matrigel; BD Biosciences) in 8-well chamber slides (Falcon), with cells suspended in propagation medium containing 5% Matrigel. The propagation medium for HMLE-derived cells was MEBM containing insulin and hydrocortisone (pituitary extract was not included) and for SUM159 cells was F12 media containing 5% FBS, insulin, and hydrocortisone. Every 4 days, the top layer was replaced with fresh propagation medium containing 20 ng/ml PDGF-BB (BD Biosciences) and 2.5% Matrigel. Cultures were maintained for 10-14 days, after which point cells were fixed and stained as previously published.

Animal Studies.

NOD/SCID mice were purchased from Jackson Laboratory. All mouse procedures were approved by the Animal Care and Use Committees of M.D. Anderson Cancer Center and performed in accordance with Institutional policies. For xenograft tumor initiation studies, the indicated number of cells were suspended in 50 μl of Matrigel diluted 1:1 with DMEM and injected into the inguinal mammary gland of NOD/SCID mice. Tumor incidence was monitored for 12 weeks following orthotopic injection. To assess the spontaneous metastatic potential of cells, 2×10⁶ HMLER-vector and HMLER-FOXC2 cells labeled with firefly luciferase were injected into the inguinal mammary gland of NOD/SCID mice. Mice were assessed weekly for metastasis via the intraperitoneal injection of D-Luciferin (Caliper LifeSciences) at 150 mg/kg in PBS, and in vivo bioluminescence was assessed using the IVIS imaging system 200 series (Xenogen Corporation). Once mammary gland tumors reached 1.5 cm in diameter, mice were euthanized and organs were harvested for confirmation of metastatic tumor burden via bioluminescence.

To ascertain the effects of sunitinib on HMLER-FOXC2 tumor growth and animal survival, 1×10⁶ firefly luciferase-labeled HMLER-FOXC2 cells were injected into the mammary fat-pad of NOD/SCID mice as described above. After 24 hours, the mice were treated 5 days a week with 40 mg/kg sunitinib or vehicle (n=9) by oral gavage. Animals were sacrificed once the tumors reached 1.5 cm³ or 30 days following injection and the dissected organs were immediately analyzed for luciferase activity.

Western Blotting and Immunofluorescence.

For western blotting, proteins were isolated by lysing cells in ice-cold radio immunoprecipitation (RIPA) buffer containing protease and phosphatase inhibitors (Roche). Protein was quantified using the Bradford Assay (BioRad) and 50 μg of total protein was resolved using 4-12% Bis-Tris SDS-PAGE gels (NuPage, Invitrogen) and transferred to PVDF membranes. Membranes were probed with primary antibodies. Following incubation with horseradish peroxidase-conjugated secondary species specific antibodies, immunoreactive proteins were detected using chemiluminescence (ECL Plus, GE Healthcare). Immunofluorescent staining of cells was performed as previously described (Mani et al., 2008). For 3D 1rECM cultures, following fixation in methanol+acetone 1:1, cells were labeled with LamininV primary antibody (Millipore Cat #AB13012) and detected with an Alexa-594 secondary antibody. Nuclei were visualized with DAPI and slides were mounted with DAKO (#S3023). All the images were acquired through an Olympus DSU spinning disc confocal microscope and analyzed at MDA Flow Cytometry and Cellular Imaging Core Facility (NCI #CA16672).

Fluorescence-Activated Cell Sorting.

The PE- and APC-conjugated anti-CD44 (clone G44-26) and FITC-conjugated anti-CD24 (clone ML5) antibodies used for FACS analysis were obtained from BD Biosciences. The PECy7-conjugated anti-CD24 (clone ML5) was purchased from BioLegend. In all instances, the antibodies were used for FACS analysis in accordance with the manufacturer's protocols. Briefly, 1×10⁶ cells in PBS+2% FBS (FACS buffer) were stained with the indicated antibodies for 30 minutes on ice. Following extensive washing, cells were resuspended in 500 μl of FACS buffer and analyzed on a BD FACSCanto II Flow Cytometer.

Quantitative Reverse Transcription PCR (qRT-PCR).

Total RNA was isolated using the RNeasy Plus kit (Qiagen) according to manufacturer's instructions. Complementary DNA was synthesized using Moloney Murine Leukemia virus reverse transcriptase (Invitrogen) following the manufacturer's instructions. The specific primer sequences used for SYBR Green qRT-PCR analysis are detailed in Table 2. Quantitative RT-PCR was performed on an Applied Biosystems 7900HT Sequence Detection System (Perkin-Elmer) equipped with a 96-well optical reaction plate. All qRT-PCR experiments were run in triplicate and a mean value was used for the determination of mRNA levels. Relative quantifications of the mRNA levels were performed using the comparative Ct method with GAPDH as the reference gene and with the formula 2^{-ΔCt}.

TABLE 2
Primers used for qRT-PCR.

| Gene | Forward Primer | Reverse Primer |
|---|---|---|
| hGAPDH | ACCCAGAAGACTGTGGATGG | TCTAGACGGCAGGTCAGGTC |
| | (SEQ ID NO: 1) | (SEQ ID NO: 2) |
| hFOXC2 | GCCTAAGGACCTGGTGAAGC | TTGACGAAGCACTCGTTGAG |
| | (SEQ ID NO: 3) | (SEQ ID NO: 4) |
| hE-cadherin (CDH1) | TGCCCAGAAAATGAAAAAGG | GTGTATGTGGCAATGCGTTC |
| | (SEQ ID NO: 5) | (SEQ ID NO: 6) |
| hN-cadherin (CDH2) | ACAGTGGCCACCTACAAAGG | CCGAGATGGGGTTGATAATG |
| | (SEQ ID NO: 7) | (SEQ ID NO: 8) |
| hFibronectin (FN1) | CAGTGGGAGACCTCGAGAAG | TCCCTCGGAACATCAGAAAC |
| | (SEQ ID NO: 9) | (SEQ ID NO: 10) |
| hVimentin (VIM) | GAGAACTTTGCCGTTGAAGC | GCTTCCTGTAGGTGGCAATC |
| | (SEQ ID NO: 11) | (SEQ ID NO: 12) |
| hSnail (SNAIL) | CCTCCCTGTCAGATGAGGAC | CCAGGCTGAGGTATTCCTTG |
| | (SEQ ID NO: 13) | (SEQ ID NO: 14) |

Transwell Migration and Invasion Assays.

Cells were serum starved for 24 hr, trypsinized, and seeded at a density of 5×10⁴ cells/well into the upper well of 24-well Transwell inserts in serum-free medium. Cells were allowed to migrate for 16 hrs using 10% FBS or PDGF-BB (20 ng/ml) in serum-free media in the lower well chamber. Following incubation, non-migrated cells were removed from the upper membrane surface and migrated cells on the lower side were fixed and stained using Diff Quick (IMEB INC Cat#K7128) and quantified by counting cells from 5 captured images per well.

Microarray Gene Expression Analysis.

Total RNA was isolated using the RNeasy Mini kit (Qiagen) from HMLER-Vector and HMLER-FOXC2 cells in triplicate samples and sent to SeqWright (Houston, Tex., USA) for sample processing and gene expression analysis using Affymetrix Human Genome U133 Plus 2.0 arrays as part of the SeqWright Gene Expression Service Suite. The resultant .CEL files were preprocessed and differential gene expression analysis performed using the Bioconductor package. To generate a FOXC2 GES, a supervised machine learning approach was used as described previously (Chang et al., 2011), where the expression values for the three vector replicates constituted the negative training set and the three FOXC2 replicates were the positive. Using the SIGNATURE platform (Chang et al., 2011), activation of the FOXC2 GES in a data set from 51 breast cancer cell lines (ArrayExpress accession E-TABM-157) (Pavon-Eternod et al., 2009) (FIG. 5D), clinical breast cancer tumors (GSE18229) (Prat et al., 2010) (FIG. 5C), and primary tumors and brain metastases for the MDA-MB-231 and CN34 xenograft models (GSE12237) (Bos et al., 2009) (FIGS. 5A and B) were scored. Using the same strategy, GES's for Twist, Gsc, Snail, and TGF-β1 mediated EMT were generated in HMLE cells (FIG. 5F) using previously described expression data (GEO accession GSE24202) (Taube et al., 2010a). As GSE18229 was generated on the Agilent platform, the probes were converted to Affymetrix by selecting the probes that target the same Entrez Gene. From this data set, only the breast tumor samples were selected, leaving 337 samples.

Chromatin Immunoprecipitation.

Chromatin immunoprecipitation (ChIP) was performed as previously described (Taube et al., 2010b). Antibodies against the following proteins were used for immunoprecipitation: normal sheep IgG (5 μA; 12-369, Upstate), FoxC2 (10 μA; N-20, Santa Cruz). To analyze specific antibody-bound DNA fractions, quantitative real time PCR was performed using Power SYBR Green (Applied Biosystems). The percentage of the input that was bound was calculated by the formula 2̂(Ct_{1% of input}−Ct_{IP}), averaged over at least three experiments, and graphed as average and standard error using GraphPad Prism v5.0 (GraphPad Software, Inc.). Subtraction of percent bound of a control IgG immunoprecipitation accounts for background of nonspecific interactions.

### Example 10

### Discussion

Many recent studies support the emerging dogma that CSCs are responsible for chemotherapy resistance, tumor relapse and metastatic competence (reviewed in May et al., 2011). CSCs and cells that have undergone EMT share many functional and molecular traits, with the corollary that engagement of the EMT program within a tumor may lead to the de novo generation and/or expansion of CSCs (Mani et al., 2008; Morel et al., 2008). Moreover, it suggests that perturbing or targeting EMT signaling pathways may provide an effective therapeutic strategy to deplete the EMT/CSC populations within a tumor. While this seems like a rational approach, the sheer number and diversity of EMT-inducing stimuli that elicit EMT in different tumor contexts will likely hinder the development of universally-applicable therapeutics. Based on the increased expression of FOXC2 following experimental induction of EMT by numerous EMT-inducing factors as well as in stem cell-enriched fractions (CD44^{high}/CD24^{low} population and mammospheres), the inventors hypothesized that FOXC2 lies at the crossroads of EMT and stem cell properties. Indeed, FOXC2 expression was found to be critical for stem cell properties, including resistance to chemotherapeutics and tumor initiation, using multiple EMT models and claudin-low breast cancer cell lines. These data clearly demonstrate that targeting FOXC2 and the associated pathways may provide an additional strategy for diminishing the CSC pool or at least those that arose via EMT.

It was surprising to see re-expression of E-cadherin in the FOXC2 depleted HMLE-Snail or TGF-β1 cells but not in Twist cells even though both Snail and Twist are continuously expressed from a retroviral expression vector and known to function similarly. While these data do not explain the failure of Snail to suppress E-cadherin expression in the absence of FOXC2, this could be mostly due to differences in epigenetic alterations.

Claudin-low tumors account for between 25% and 39% of triple-negative breast cancers (ER⁻/PR⁻/HER2⁻); shown to resemble most closely with mammary epithelial stem cells; and are also enriched for markers of EMT and CSCs (Prat et al., 2010; Taube et al., 2010a). The enrichment of FOXC2 expression and its associated GES in claudin-low tumors (Creighton et al., 2009; Li et al., 2008; Herschkowitz et al., 2007; Prat et al., 2010) provides the first evidence that FOXC2 transcriptional activity may play an important functional role for this molecular subtype.

Targeting the FOXC2 pathway may be an effective therapeutic strategy for tumors with enriched EMT/CSC properties. Future studies to assess the protein expression of FOXC2 pathway members in clinical specimens will be critical. As transcription factors can be difficult to directly inhibit therapeutically, a potential target for FOXC2-expressing tumors is PDGFR-β, which has FDA approved small molecule inhibitors, such as sunitinib. However, as sunitinib is a multi-targeted tyrosine kinase inhibitor, future studies using more specific pharmacological inhibitors of PDGFR-β or RNAi approaches will be required to determine if PDGFR-β is a key functional mediator of FOXC2 in EMT-derived cells and claudin-low tumors. Nevertheless, sunitinib and other PDGFR inhibitors may be effective in patients with claudin-low or therapy-resistant tumors displaying elevated FOXC2 expression.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,801,005
U.S. Pat. No. 5,739,169
U.S. Pat. No. 5,830,880
U.S. Pat. No. 5,846,945
U.S. Pat. No. 5,824,311
U.S. Pat. No. 5,760,395
U.S. Pat. No. 4,870,287
WO 98/54216
WO 01/60853
Abraham et al., Prevalence of CD44+/CD24−/low cells in breast cancer may not be associated with clinical outcome but may favor distant metastasis. Clin. Cancer Res., 11:1154-1159, 2005.
Al-Hajj et al., Prospective identification of tumorigenic breast cancer cells. Proc. Natl. Acad. Sci. U.S.A., 100:3983-3988, 2003.
Ausubel et al., 2003.
Battula et al., Epithelial-mesenchymal transition-derived cells exhibit multilineage differentiation potential similar to mesenchymal stem cells. Stem Cells, 28:1435-1445, 2010.
Blanco et al., Correlation of Snail expression with histological grade and lymph node status in breast carcinomas. Oncogene, 21:3241-3246, 2002.
Bos et al., Genes that mediate breast cancer metastasis to the brain. Nature, 459:1005-1009, 2009.
Bukowski et al., Signal transduction abnormalities in T lymphocytes from patients with advanced renal carcinoma: clinical relevance and effects of cytokine therapy. Clin. Cancer Res., 4:2337-2347, 1998.
Chang et al., SIGNATURE: a workbench for gene expression signature analysis. BMC Bioinformatics, 12:443, 2011.
Christodoulides et al., Immunization with recombinant class 1 outer-membrane protein from Neisseria meningitidis: influence of liposomes and adjuvants on antibody avidity, recognition of native protein and the induction of a bactericidal immune response against meningococci. Microbiology, 144:3027-3037, 1998.
Creighton et al., Residual breast cancers after conventional therapy display mesenchymal as well as tumor-initiating features. Proc. Natl. Acad. Sci. U.S.A., 106:13820-13825, 2009.
Davidson et al., Intralesional cytokine therapy in cancer: a pilot study of GM-CSF infusion in mesothelioma. J. Immunother., 21:389-398, 1998.
Dontu et al., In vitro propagation and transcriptional profiling of human mammary stem/progenitor cells. Genes Dev., 17:1253-1270, 2003.
Fillmore et al., Human breast cancer cell lines contain stem-like cells that self-renew, give rise to phenotypically diverse progeny and survive chemotherapy. Breast Cancer Res., 10:R25, 2008.
Ginestier et al., ALDH1 Is a Marker of Normal and Malignant Human Mammary Stem Cells and a Predictor of Poor Clinical Outcome. Cell Stem Cell, 1:555-567, 2007.
Gupta et al., Identification of selective inhibitors of cancer stem cells by high-throughput screening. Cell, 138:645-659, 2009.
Hanibuchi et al., Therapeutic efficacy of mouse-human chimeric anti-ganglioside GM2 monoclonal antibody against multiple organ micrometastases of human lung cancer in NK cell-depleted SCID mice. Int. J. Cancer, 78:480-485, 1998.
Hay, An overview of epithelio-mesenchymal transformation. Acta Anat (Basel) 1995; 154: 8-20.
Hellstrand et al., Histamine and cytokine therapy. Acta Oncol., 37:347-353, 1998.
Herschkowitz et al., Identification of conserved gene expression features between murine mammary carcinoma models and human breast tumors. Genome Biol., 8:R76, 2007.
Hollander, Immunotherapy for B-cell lymphoma: current status and prospective advances. Front. Immunol., 3:3, 2012.
Hollier et al., The epithelial-to-mesenchymal transition and cancer stem cells: a coalition against cancer therapies. J. Mammary Gland Biol. Neoplasia, 14:29-43, 2009.
Huber et al., Molecular requirements for epithelial-mesenchymal transition during tumor progression. Curr. Opin. Cell Biol., 17:548-558, 2005.
Hui and Hashimoto, Pathways for potentiation of immunogenicity during adjuvant-assisted immunizations with Plasmodium falciparum major merozoite surface protein 1. Infect. Immun., 66:5329-5336, 1998.
Kenny et al., The morphologies of breast cancer cell lines in three-dimensional assays correlate with their profiles of gene expression. Mol. Oncol. , 1:84-96, 2007.
Kim et al., Constitutively active type I insulin-like growth factor receptor causes transformation and xenograft growth of immortalized mammary epithelial cells and is accompanied by an epithelial-to-mesenchymal transition mediated by NF-kappaB and snail. Mol. Cell. Biol., 27:3165-3175, 2007.
Lee et al., Three-dimensional culture models of normal and malignant breast epithelial cells. Nat. Methods, 4:359-365, 2007.
Li et al., Intrinsic resistance of tumorigenic breast cancer cells to chemotherapy. J. Natl. Cancer Inst., 100:672-679, 2008.
Liu et al., The prognostic role of a gene signature from tumorigenic breast-cancer cells. N. Engl. J. Med., 356:217-226, 2007.
Mani et al., Mesenchyme Forkhead 1 (FOXC2) plays a key role in metastasis and is associated with aggressive basal-like breast cancers. Proc. Natl. Acad. Sci. U.S.A., 104:10069-100074, 2007.
Mani et al., The epithelial-mesenchymal transition generates cells with properties of stem cells. Cell, 133:704-715, 2008.
May et al., Epithelial-mesenchymal transition and cancer stem cells: a dangerously dynamic duo in breast cancer progression. Breast Cancer Res., 13:202, 2011.
Miura et al., FEBS Letters, 326:171-176. 1993.
Miura et al., Genomics, 41:489-492, 1997.
Morel et al., Generation of breast cancer stem cells through epithelial-mesenchymal transition. PLoS One, 3:e2888.
Moustakas et al., Signaling networks guiding epithelial-mesenchymal transitions during embryogenesis and cancer progression. Cancer Sci., 98:1512-1520, 2007.
Muthuswamy et al., ErbB2, but not ErbB1, reinitiates proliferation and induces luminal repopulation in epithelial acini. Nat. Cell Biol., 3:785-792, 2001.
Pavon-Eternod et al., tRNA over-expression in breast cancer and functional consequences. Nucleic Acids Res., 37:7268-7280, 2009.
Petersen et al., Interaction with basement membrane serves to rapidly distinguish growth and differentiation pattern of normal and malignant human breast epithelial cells. Proc. Natl. Acad. Sci. U.S.A., 89:9064-9068, 1992.
Prat et al., Phenotypic and molecular characterization of the claudin-low intrinsic subtype of breast cancer. Breast Cancer Res., 12:R68, 2010.
Qin et al., Interferon-beta gene therapy inhibits tumor formation and causes regression of established tumors in immune-deficient mice. Proc. Natl. Acad. Sci. U.S.A., 95:14411-14416, 1998.
Sambrook et al., 1989.
Savagner et al., Modulations of the epithelial phenotype during embryogenesis and cancer progression. Cancer Treat Res., 71:229-249, 1994.
Shackleton et al., Heterogeneity in cancer: cancer stem cells versus clonal evolution. Cell, 138:822-829, 2009.
Sheridan et al., CD44+/CD24− breast cancer cells exhibit enhanced invasive properties: an early step necessary for metastasis. Breast Cancer Res., 8:R59, 2006.
Stewart et al., Lentivirus-delivered stable gene silencing by RNAi in primary cells. RNA, 9:493-501, 2003.
Taube et al., Core epithelial-to-mesenchymal transition interactome gene-expression signature is associated with claudin-low and metaplastic breast cancer subtypes. Proc. Natl. Acad. Sci. U.S.A., 107:15449-15454, 2010a.
Taube et al., Foxa1 functions as a pioneer transcription factor at transposable elements to activate Afp during differentiation of embryonic stem cells. J. Biol. Chem., 285:16135-16144, 2010b.
Thiery, Epithelial-mesenchymal transitions in tumour progression. Nat. Rev. Cancer, 2:442-454, 2002.
Thiery, Epithelial-mesenchymal transitions in development and pathologies. Curr. Opin. Cell. Biol., 15:740-746, 2003.
Thiery et al., Complex networks orchestrate epithelial-mesenchymal transitions. Nat. Rev. Mol. Cell. Biol., 7:131-142, 2006.
Ugolini et al., WNT pathway and mammary carcinogenesis: loss of expression of candidate tumor suppressor gene SFRP1 in most invasive carcinomas except of the medullary type. Oncogene, 20:5810-5817, 2001.
Viloria-Petit et al., A role for the TGFbeta-Par6 polarity pathway in breast cancer progression. Proc. Natl. Acad. Sci. U.S.A., 106:14028-14033, 2009.
Woodward et al., WNT/beta-catenin mediates radiation resistance of mouse mammary progenitor cells. Proc. Natl. Acad. Sci. U.S.A., 104:618-623, 2007.
Yang et al., Twist, a master regulator of morphogenesis, plays an essential role in tumor metastasis. Cell, 117:927-939, 2004.
Yang et al., Epithelial-mesenchymal transition: at the crossroads of development and tumor metastasis. Dev. Cell, 14:818-829, 2008.
Yu et al., let-7 regulates self renewal and tumorigenicity of breast cancer cells. Cell, 131:1109-1123, 2007.

What is claimed is:

1. A method of treating a patient comprising:
(a) selecting a patient determined to comprise cancer stem cells; and
(b) treating the patient with an effective amount of a PDGFR-β inhibitor, thereby inhibiting the cancer stem cells.

2. The method of claim 1, wherein the tumor is a breast cancer, colon cancer, prostate cancer, or a brain tumor.

3. The method of claim 1, wherein selecting a patient determined to comprise cancer stem cells comprises selecting a patient determined to have an elevated level of FOXC2 or PDGFR-β relative to a reference level.

4. The method of claim 2, wherein the breast cancer is a Claudin-low breast cancer.

5. The method of claim 1, wherein the patient has a metastatic cancer.

6. The method of claim 1, wherein the PDGFR-β inhibitor is sunitinib, axitinib, BIBF1120, MK-2461, dovitinib, pazopanib, telatinib, CP 673451, or TSU-68.

7. The method of claim 1, wherein the PDGFR-β inhibitor is administered in conjunction with at least a second therapy.

8. The method of claim 1, wherein the patient has previously undergone at least one round of anti-cancer therapy.

9. The method of claim 1, wherein the patient is in cancer remission.

10. The method of claim 1, wherein (a) selecting a patient comprises selecting a patient determined to comprise cancer cells that express an elevated level of FOXC2 or PDGFR-β relative to a reference level.

11. A method of predicting sensitivity of a cancer in a patient to PDGFR-β inhibitors comprising:
(a) obtaining a sample of the cancer; and
(b) assaying to determine a level of FOXC2 or PDGFR-β expression in the sample, wherein if the level of FOXC2 or PDGFR-β is elevated relative to a reference level, then the cancer is predicted to be sensitive to a PDGFR-β inhibitor or wherein if the level of FOXC2 or PDGFR-β is not elevated relative to a reference level, then the cancer is not predicted to be sensitive to a PDGFR-β inhibitor.

12. The method of claim 11, further comprising assaying to determine the expression level of FOXC2 and PDGFR-β.

13. The method of claim 11, further comprising:
(c) identifying the patient as having a cancer that is sensitive to a PDGFR-β inhibitor if the level of FOXC2 or PDGFR-β is elevated relative to a reference level; or identifying the patient as having a cancer that is not sensitive to a PDGFR-β inhibitor if the level of FOXC2 or PDGFR-β is not elevated relative to a reference level.

14. The method of claim 13, wherein identifying comprises reporting whether the patient has a cancer that is sensitive to a PDGFR-β inhibitor.

15. The method of claim 11, wherein assaying to determine a level of FOXC2 or PDGFR-β expression comprises determining a level of FOXC2 or PDGFR-β protein expression.

16. The method of claim 15, wherein assaying comprises measuring the amount of FOXC2 or PDGFR-β protein in the sample to the amount of FOXC2 or PDGFR-β protein in a control sample by contacting the samples to an antibody that binds to FOXC2 or PDGFR-β and comparing the amount of protein in the sample and the control sample.

17. The method of claim 11, wherein assaying to determine a level of FOXC2 or PDGFR-β expression comprises determining a level of FOXC2 or PDGFR-β RNA expression.

18. The method of claim 17, wherein assaying comprises measuring the amount of FOXC2 or PDGFR-β RNA in the sample to the amount of FOXC2 or PDGFR-β RNA in a control sample by hybridization of samples with a nucleic acid molecule that binds to FOXC2 or PDGFR-β RNA and comparing the amount of RNA in the sample and the control sample.

19. A method of treating a patient comprising:
(a) selecting a patient determined to comprise cancer stem cells that are sensitive to PDGFR-β inhibitors in accordance with claim 11; and
(b) treating the patient with an effective amount of a PDGFR-β inhibitor, thereby inhibiting the cancer stem cells.

20. A method of monitoring the efficacy of PDGFR-β treatment on a cancer comprising:
(a) obtaining samples of the cancer from at least two time points during the course of treatment;
(b) assaying the FOXC2 or PDGFR-β expression level in the samples; and
(c) comparing the FOXC2 or PDGFR-β expression levels, wherein the PDGFR-β pathway inhibitor treatment is efficacious if the FOXC2 or PDGFR-β expression level decreases over the course of treatment.
